(19) United States
(12) Patent Application Publication
(10) Pub. No.: US 2022/0177521 A1
(43) Pub. Date: Jun. 9, 2022

(54) PROCESS FOR THE PREPARATION OF DEGARELIX

(71) Applicant: FRESENIUS KABI IPSUM S.R.L., Cassina de' Pacchi (IT)

(72) Inventors: Walter Cabri, Cassina de' Pacchi (IT); Andrea Orlandin, Cassina de' Pacchi (IT); Angelo Viola, Cassina de' Pacchi (IT); Antonio Ricci, Cassina de' Pacchi (IT); Ivan Guryanov, Cassina de' Pacchi (IT)

(21) Appl. No.: 17/437,019

(22) PCT Filed: Mar. 5, 2020
(86) PCT No.: PCT/EP2020/055895

§ 371 Date: Sep. 7, 2021

(30) Foreign Application Priority Data
Mar. 7, 2019 (EP) 19161404.9

(51) Int. Cl. C07K 7/23

(52) U.S. Cl. CPC C07K 7/23

(57) ABSTRACT

The present invention provides a manufacturing process for the preparation of degarelix by using Fmoc protected amino acids as building blocks, wherein the Fmoc group is cleaved by treatment with tert-butylamine.

### FIELD OF THE INVENTION

The present invention relates to peptide synthesis. In particular, it relates to a process for the preparation of decapeptide degarelix by using Fmoc protected amino acids as building blocks.

### BACKGROUND OF THE INVENTION

The synthesis of peptides carrying at least one p-amino-phenylalanine (Aph) derivative, such as for example Aph(Hor), Aph(Cbm) or Aph(Atz) in their amino acid sequence is challenging. The synthesis often results in a product with a high amount of impurities (such as deletion products or products of side reactions).

The most prominent example of such a peptide is degarelix (I), a decapeptide (ten amino acids) approved as a medicinal product for the treatment of patients with advanced prostate cancer and marketed under the trade name Firmagon®, as a third-generation gonadotropin releasing hormone (GnRH) receptor antagonist (a GnRH blocker).

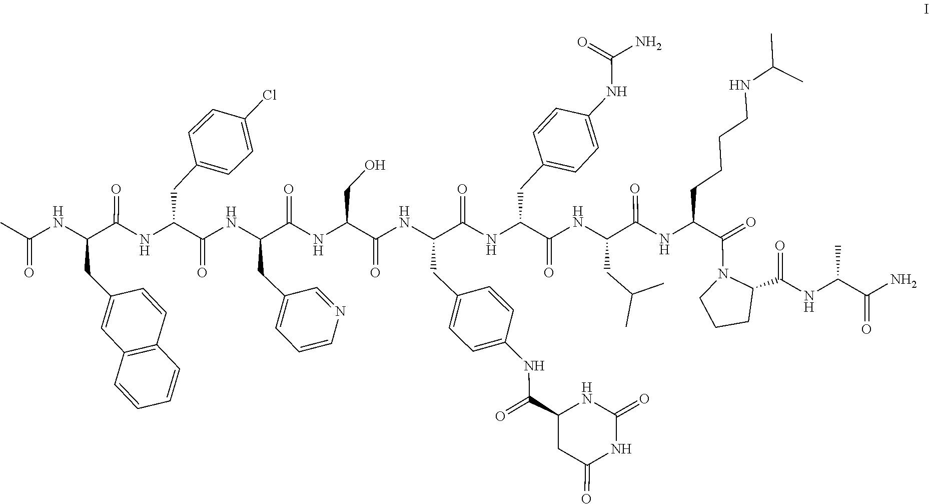 [SMILES: CC(=O)N[C@H](Cc1ccc2ccccc2c1)C(=O)N[C@H](Cc1ccc(Cl)cc1)C(=O)N[C@H](Cc1cccnc1)C(=O)N[C@@H](CO)C(=O)N[C@@H](Cc1ccc(NC(=O)[C@@H]2CC(=O)NC(=O)N2)cc1)C(=O)N[C@H](Cc1ccc(NC(N)=O)cc1)C(=O)N[C@@H](CC(C)C)C(=O)N[C@@H](CCCCNC(C)C)C(=O)N1CCC[C@H]1C(=O)N[C@H](C)C(N)=O]

Degarelix is also identified by the sequence:

```
            1 5
Ac-D-Nal-D-Cpa-D-Pal-Ser-Aph(Hor)-D-Aph(Cbm)-Leu-
                                10
Lys(iPr)-Pro-D-Ala-NH₂
```

wherein the numbers indicate the amino acid (aa) positions, starting from N-terminal aa (D-Nal) to C-terminal aa (D-Ala).

Due to many advantages over other GnRH antagonists, degarelix became widely used for the treatment of advanced prostate cancer (M. Steinberg, Clin. Therapeutics, 2009, 31, 2312-2331). The presence of unnatural amino acids, which are susceptible for rearrangements and side reactions, in the structure of degarelix complicates its chemical synthesis using the conventional methods of peptide chemistry.

One of the main problems in the preparation of degarelix is the high sensitivity of the (L)dihydroorotic acid (indicated as Hor) moiety of the Aph(Hor) residue in position 5 of the sequence in the presence of an aqueous basic solution. Under these conditions, a rapid rearrangement of the 6-membered Hor ring occurs, with intermediate hydrolysis to an N-carbamoyl-aspartyl fragment followed by formation of a 5-membered hydantoin (dihydroorotyl-hydantoin rearrangement, Scheme 1) (see also J. Kaneti, A. J. Kirby, A. H. Koedjikov and I. G. Pojarlieff, Org. Biomol. Chem. 2004, 2, 1098-1103).

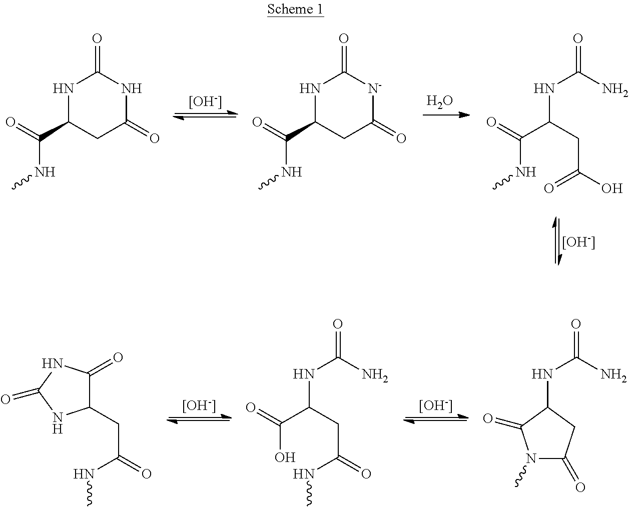

The hydantoin-degarelix impurity (II) formed through such rearrangement has a high structure similarity to degarelix, therefore its presence may noticeably complicate the downstream process for the completion of the peptide preparation, in particular the purification step. Even a small amount of such an impurity may drastically decrease the final yield of the preparation process.

It has been reported that this problem occurs during the synthesis of degarelix using 9-fluorenylmethyloxycarbonyl (Fmoc) protected amino acids as building blocks, as repeated Fmoc deprotection cycles in basic conditions are involved.

WO2010121835, for instance, disclosed that the treatment of degarelix with 1,8-diazabicyclo[5.4.0]-undec-7-ene (DBU) 2% solution in DMF resulted in the formation of 1.8% of the hydantoin-degarelix impurity (II). The amount of such impurity increased up to 7%, when 5% water was added to the basic solution. Nevertheless, piperidine 20% solution in DMF, employed as standard Fmoc cleavage reagent, was stated to reduce formation of the hydantoin-degarelix impurity (II) to not more than 0.3% by weight.

WO2017103275 disclosed a synthesis of degarelix with Fmoc SPPS, characterized by the incorporation of p-nitro-phenylalanine (indicated as Phe(NO₂)) at position 5 of the sequence and its subsequent transformation into Aph(Hor) first by reduction of the nitro group and then by coupling with Hor or a derivative thereof.

As above explained, the possibility of dihydroorotic moiety rearrangement during peptide synthesis in the presence of bases significantly limits the choice of the deprotection mixtures and, therefore, the applicability of Fmoc-based protection in the preparation of degarelix remains a challenge.

Accordingly, there remains a need to develop an efficient, simple and industrially viable synthetic process for the preparation of degarelix, which can overcome the drawbacks of the prior art and which provides the crude peptide in high yield and a favorable impurity profile, facilitating final purification and improving final yield.

### SUMMARY OF THE INVENTION

The present invention provides a process for the preparation of degarelix, or a pharmaceutically acceptable salt thereof, by using Fmoc protected amino acids as building blocks, characterized in that the Fmoc group is cleaved by treatment with tert-butylamine.

The present invention further provides a process for the preparation of degarelix, or a pharmaceutically acceptable salt thereof, through peptide solid phase synthesis (SPPS) by using Fmoc protected amino acids as building blocks, characterized in that the Fmoc group is cleaved by treatment with tert-butylamine.

Moreover, the present invention provides a process for the preparation of degarelix, or a pharmaceutically acceptable salt thereof, through SPPS by using Fmoc protected amino acids comprising Fmoc-Phe(NO₂)—OH as building block, characterized in that the Fmoc group is cleaved by treatment with tert-butylamine.

The present invention further provides a process for the preparation of degarelix, or a pharmaceutically acceptable salt thereof, wherein degarelix comprises 0.15% by weight or less of hydantoin-degarelix impurity (II).

### DESCRIPTION OF FIGURES

FIG. 1: Graphical representation of the different Fmoc cleavage rates from an Fmoc-Phe(NO₂)-Rink Amide resin in the presence of four different bases, piperidine, N-methylpiperazine, morpholine and tert-butylamine.

FIG. 2: Graphical representation of the different Fmoc cleavage rates from an Fmoc-Rink Amide resin in the presence of different concentration of TBA in DMF.

FIG. 3: Graphical representation of the different Fmoc cleavage rates from an Fmoc-Ser(tBu)-Rink Amide resin in the presence of different concentration of TBA in DMF.

All graphs in the Figures depict the Fmoc removal % vs. time(min). (T.C.F.D. stands for Theoretical Complete Fmoc Deprotection).

### DETAILED DESCRIPTION OF THE INVENTION

A hydantoin-degarelix impurity (II) may be formed through the dihydroorotyl-hydantoin rearrangement as depicted in Scheme 1, in the presence of an aqueous basic solution.

Such impurity has the chemical structure shown below:

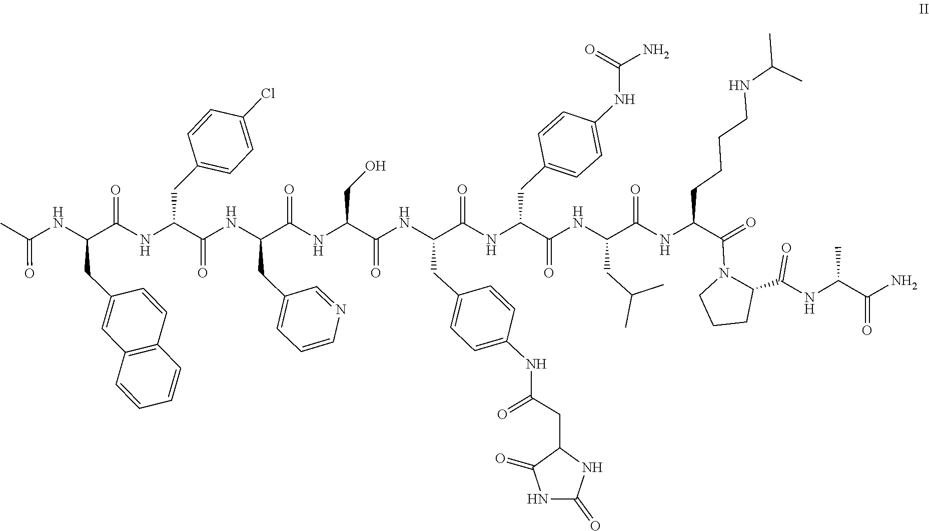 [SMILES: CC(=O)N[C@H](Cc1ccc2ccccc2c1)C(=O)N[C@H](Cc1ccc(Cl)cc1)C(=O)N[C@H](Cc1cccnc1)C(=O)N[C@@H](CO)C(=O)N[C@@H](Cc1ccc(NC(=O)CC2NC(=O)NC2=O)cc1)C(=O)N[C@H](Cc1ccc(NC(N)=O)cc1)C(=O)N[C@@H](CC(C)C)C(=O)N[C@@H](CCCCNC(C)C)C(=O)N1CCC[C@H]1C(=O)N[C@H](C)C(N)=O]

Alternatively, such impurity is also indicated as

(II)
Ac-D-Nal-D-Cpa-D-Pal-Ser-Aph(Hyd)-D-Aph(Cbm)-Leu-
Lys(iPr)-Pro-D-Ala-NH₂

wherein Hyd indicates 4-([2-(5-hydantoyl)]acetyl.

When looking for an efficient, simple and industrially viable synthetic process for the preparation of degarelix, which can overcome the drawbacks of the prior art and results in an even lower formation of the hydantoin impurity than when piperidine is used in an Fmoc based SPPS synthesis, one would search for a base structurally similar to piperidine, such as pyrrolidine, N-methylpiperazine, morpholine and DBU.

To test these bases degarelix was dissolved in a mixture of N,N-dimethylformamide (DMF) and different bases, and its stability was tested by HPLC over time by sampling at specific time intervals, i.e. at 20 min (duration of a standard Fmoc deprotection cycle), at 1 hour 40 min (5 standard Fmoc deprotection cycles, needed to attach 4 amino acids after 5-Aph(Hor) residue and to finally acetylate the peptide at N-terminal), and at 20 hours. It turned out that treatment with pyrrolidine, N-methyl-piperazine and morpholine resulted in significantly lower hydantoin formation rates than with piperidine or DBU.

When, further experiments were performed to test the rearrangement rate of the orotyl residue in these different bases in the presence of water (employing the same mixture of DMF and base, but with the addition of 5% water) it became clear that these bases remained amongst the best performers.

The strongest base with the highest pKₐ, DBU, favored the rearrangement even in the absence of water.

However, even though these bases resulted in a favorably low hydantoin formation rate, an increase of other impurities was observed, especially for pyrrolidine, which can only be explained by a significant rate of degradation of degarelix.

Surprisingly, it was then found that when degarelix was treated with tert-butylamine, a primary amine, structurally unrelated to piperidine or DBU, no hydantoin-degarelix impurity (II) was formed and no significant increase in other impurities could be determined.

Without wishing to be bound by theory, it is believed that sterical hindrance by tert-butylamine may prevent the deprotonation of dihydroorotic moiety at the first step of the process of isomerization.

The experimental details are reported in the Examples section (Example 1).

TABLE 1
Stability of degarelix vs. dihydroorotyl-hydantoin rearrangement in the presence
of different amines

| | | | | | Hydantoin- |
|---|---|---|---|---|---|
| | | | | | degarelix |
| | | | Monitored | Hydantoin- | impurity (II) in |
| | | base in | Time | degarelix | the presence of |
| Base Name | Base Structure | DMF, % | Intervals | impurity (II), % | 5% water, % |
| DBU | 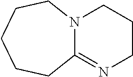 [SMILES: C1CCC2=NCCCN2CC1] | 2 | 20 min 1 h 40 min 20 h | <0.10 0.69 4.97 | <0.10 1.38 10.14 |
| Piperidine | 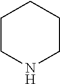 | 20 | 20 min 1 h 40 min 20 h | <0.10 0.16 0.18 | 0.22 0.26 0.67 |
| Pyrrolidine | 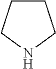 | 20 | 20 min 1 h 40 min 20 h | <0.10 <0.10 <0.10 | <0.10 <0.10 <0.10 |
| N-methyl piperazine | 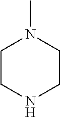 | 5 | 20 min 1 h 40 min 20 h | <0.10 <0.10 <0.10 | <0.10 <0.10 <0.10 |
| Morpholine | 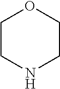 | 50 | 20 min 1 h 40 min 20 h | <0.10 <0.10 <0.10 | <0.10 <0.10 <0.10 |
| tert-butylamine (TBA) | 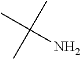 | 30 | 20 min 1 h 40 min 20 h | <0.10 <0.10 <0.10 | <0.10 <0.10 0.43 |

To confirm whether tert-butylamine indeed was suited as base for the Fmoc based SPPS of degarelix, a second set of experiments was carried out to test the kinetics of Fmoc deprotection with tert-butylamine in comparison to other bases on a suitable model substrate. Namely, Fmoc-Phe(NO₂)—OH, attached to Rink amide resin as the solid support, was used and the Fmoc cleavage rates for the cyclic secondary amines piperidine, N-methylpiperazine and morpholine, were compared to that of the primary non-cyclic amine tert-butylamine. The experimental details are reported in the Examples section (Example 2).

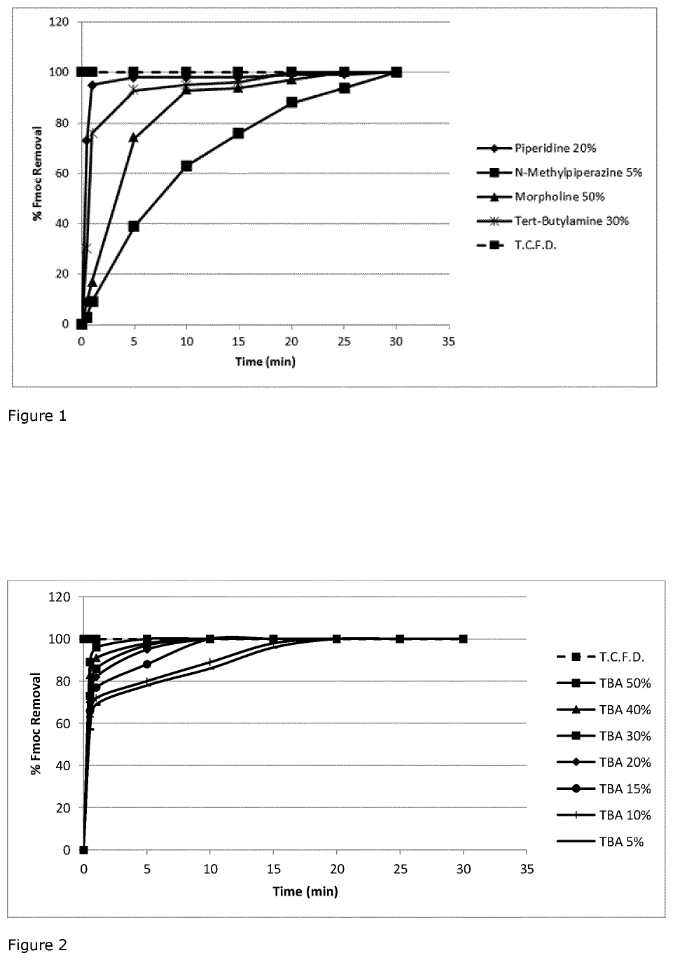

The experimental results reported in FIG. 1 showed that surprisingly Fmoc deprotection kinetics using tert-butylamine were comparable to that performed with piperidine. In fact, piperidine and tert-butylamine, induced almost complete Fmoc cleavage in a few minutes.

On the contrary, the morpholine and N-methylpiperazine could remove the Fmoc protective group only much more slowly on the model amino acid.

The same pattern was observed when Fmoc-protected Rink amide resin and Fmoc-Ser(tBu)-Rink amide resin were used as models (data not shown).

A slower Fmoc removal rate may favor the formation of truncated sequences in case Fmoc deprotection is not complete before the attachment of the next amino acid in the sequence.

Surprisingly therefore, tert-butylamine, also referred to as TBA, showed to have the best combination of rapidly cleaving the Fmoc group and minimizing dihydroorotyl-hydantoin rearrangement over prolonged time period.

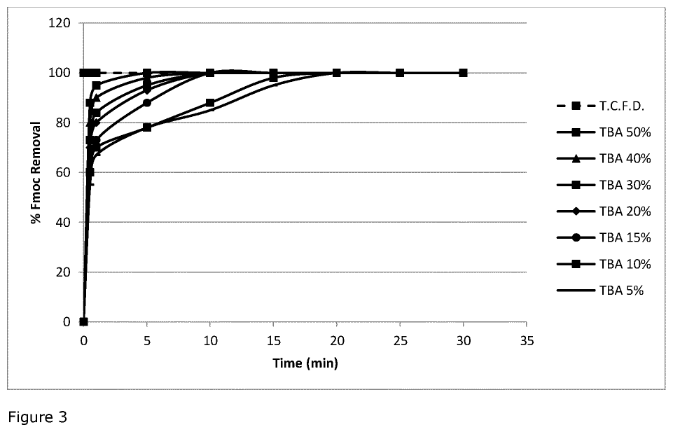

Further experiments were performed onto two model substrates to test the range of TBA concentration that can be used to efficiently carry out the Fmoc cleavage step. The results are shown in FIGS. 2 and 3, and the experimental details are reported in the Examples section (Example 5). TBA concentration can vary from 5 to 50% obtaining 100% Fmoc protection in reasonable time, i.e. within 20 min. The use of tert-butylamine was then tested in the preparation of degarelix in solid phase both by stepwise SPPS and by incorporation of 5-Phe(NO₂) in a degarelix intermediate, followed by nitro group reduction and by coupling with (L)dihydroorotic acid, according to the approach described in example 2 of WO2017103275.

Purity of the crude peptides and presence of hydantoin-degarelix impurity (II) in same crude were tested by HPLC. The results are reported in Table 2, where HPLC % purity and HPLC % hydantoin-degarelix impurity (II) are shown.

TABLE 2

| Strategy of SPPS degarelix | | Hydantoin-degarelix |
|---|---|---|
| preparation, base | HPLC purity, % | impurity (II), % |
| Stepwise, TBA | 87.5 | <0.15 |
| 5-Phe(NO₂)-degarelix reduction | 88.6 | <0.15 |
| and Hor coupling, TBA | | |
| Prior Art (WO2010121835) | n.a. | <0.3 |
| Stepwise, piperidine | | |

n.a. not available

The present invention thus provides a process for the preparation of degarelix (I), or a pharmaceutically acceptable salt thereof, by using Fmoc protected amino acids as building blocks, characterized in that the Fmoc group is cleaved by treatment with tert-butylamine.

Such preparation can be carried out by standard peptide synthesis techniques such as Liquid Phase Peptide Synthesis (LPPS) and Solid Phase Peptide Synthesis (SPPS). In particular, the preparation in solid phase can be carried out as a stepwise—or sequential—SPPS, wherein the amino acids are coupled one by one to the growing peptide sequence attached to a solid support, or as a Convergent SPPS (CSPPS), wherein at least two peptide fragments, independently prepared, are coupled together to form amide bonds and longer peptide fragments, until the final sequence is finally obtained, wherein one of the two fragments involved in a coupling reaction is attached to a solid support.

The terms “peptide”, “peptide fragment” and “fragment”, as used herein, describe a partial sequence of amino acids, with a minimum length of 2 amino acids, with reference to the degarelix sequence. It can be optionally attached to a resin at its C-terminal amino acid. A peptide fragment can be protected or not protected.

The terms “protected peptide fragment” or “protected fragment” describe a peptide fragment which can independently bear protecting groups at its amino acids side-chains, or side groups, and/or at its alpha-amino group.

The term “nitro-peptide”, as used herein, is a peptide as defined above, comprising one or two p-nitro-phenylalanine residues.

The terms “resin” or “solid support” describes a functionalized insoluble polymer to which an amino acid or a peptide fragment can be attached and which is suitable for amino acids elongation until the full desired sequence is obtained.

More specifically, stepwise SPPS can be defined as a process in which a peptide anchored by its C-terminal amino acid to a solid support, i.e. a resin, is assembled by the sequential addition of the optionally protected amino acids constituting its sequence. It comprises the loading of a first alpha-amino-protected amino acid, or peptide, or pseudoproline dipeptide, onto a resin, followed by the repetition of a sequence of steps referred to as a cycle, or as a step of elongation, consisting of the cleavage of the alpha-amino protecting group and the coupling of the subsequent protected amino acid.

The formation of a peptide bond between two amino acids, or between an amino acid and a peptide fragment, or between two peptide fragments, also indicated as coupling reaction, may involve two steps. First, the optional activation of the free carboxyl group for a time ranging from 5 minutes to 2 hours, then the nucleophilic attack of the free amino group at the activated carboxylic group.

The cycle as defined above may be repeated until the desired sequence of the peptide is accomplished.

Finally, the peptide is deprotected and/or cleaved from the resin.

As a reference for SPPS, please see for instance Knud J. Jensen et al. (eds.), Peptide Synthesis and Applications, Methods in Molecular Biology, vol. 1047, Springer Science, 2013.

In a preferred aspect of present invention, in the preparation of degarelix a resin is used which is selected from the group consisting of Rink amide, Rink amide AM, Rink amide MBHA, Wang, 2-chlorotrityl chloride (CTC) and trityl chloride resin.

Rink amide, Rink amide AM resin and Rink amide MBHA resin have the advantage that they allow obtaining directly a C-terminal amide after cleavage of the peptide from the resin, therefore they are particularly suitable for the preparation of degarelix.

More preferably, in the process of the present invention Rink amide resin is used; even more preferably, Fmoc-protected Rink amide resin (Fmoc Rink amide resin).

In a preferred aspect of present invention, the loading of the first C-terminal amino acid, i.e. D-Alanine, onto the resin is carried out by swelling the resin in a suitable solvent, preferably DMF, filtering the resin and adding to the resin a solution of the Fmoc protected amino acid with an activating agent, such as a carbodiimide, for instance DIC.

In case, a Fmoc-protected solid support is used, as for instance Fmoc Rink amide resin, before loading the first C-terminal amino acid, Fmoc group needs to be cleaved, and any suitable base can be used. In some embodiments, the Fmoc protecting group is cleaved by treatment with an amine selected from the group consisting of piperidine, pyrrolidine, piperazine, DBU and tert-butylamine.

In a certain aspect of the present invention, after the first C-terminal amino acid has been loaded onto the resin, an additional step to block unreacted sites is optionally performed to avoid truncated sequences and to prevent any side reactions. Such step is often referred to as “capping”.

Capping is achieved by a short treatment of the loaded resin with a large excess of a highly reactive unhindered reagent, which is chosen according to the unreacted sites to be capped. Optionally, capping is performed in basic conditions, for instance in the presence of DIPEA. When using a Wang resin, the unreacted sites are hydroxyl groups, which are preferably capped by treatment with an acid derivative, such as an anhydride, for instance with Ac₂O. When using a CTC resin, the unreacted sites are chlorines, which are preferably capped by treatment with an alcohol, for instance with MeOH in a basic medium, like for instance with a DCM/DIPEA/MeOH mixture. Then, after washing with DCM, the resin is further treated with an Ac₂O mixture, to cap the hydroxyl groups possibly resulting from the chlorine hydrolysis. When using a Rink Amide resin, similarly capping can be performed for instance by using an Ac₂O mixture, like for instance a DMF/Ac₂O, optionally in the presence of DIPEA.

A similar capping procedure is optionally performed also after each coupling reaction to block the unreacted amino groups. Such procedure would also avoid truncated sequence and is substantially similar to the capping performed after loading of the first amino acid, and can be performed for instance by using a DMF/Ac₂O mixture.

As an alternative to the loading of the first C-terminal amino acid, preloaded resins are used in the preparation of peptide fragments. These are commercially available Rink amide/Wang/CTC resins with attached Fmoc-protected L- or D-amino acids. Accordingly, for instance, Fmoc-D-Ala-Rink amide resin is preferably used for the synthesis of degarelix.

In a preferred aspect of present invention, the loading of the first C-terminal amino acid onto the resin is determined spectrophotometrically, as described for instance in Knud J. Jensen et al. (eds.), Peptide Synthesis and Applications, Methods in Molecular Biology, vol. 1047, Springer Science, 2013.

In a preferred aspect of the present invention, each amino acid may be protected at its alpha-amino group and/or at its side-chain functional groups.

The protecting group for the amino acids alpha-amino groups that is used in the process of the present invention is of the 9-fluorenylmethoxycarbonyl (Fmoc) type, and it is removed, or cleaved, by treatment with tert-butylamine.

The amino acids side-chain functional groups are optionally protected with groups which are generally stable during coupling reactions and during alpha-amino protecting group removal, and which are themselves removable in suitable conditions. The protecting groups of amino acids side-chain functional groups which are used in the present disclosure are generally removable in acidic conditions, as orthogonal to the basic conditions used to deprotect Fmoc protecting groups, i.e. such protecting groups are stable to the treatment with tert-butylamine.

In a preferred aspect of the present invention, such side-chain protecting groups (PG) are specified per individual amino acid occurring in degarelix sequence, as follows:

the hydroxyl group of serine (Ser) is preferably protected by a PG selected from the group consisting of trityl (Trt), tertbutyldimethylsilyl (TBDMS) and tertbutyl (tBu); more preferably, the tBu group is used;

the s-amino group of lysine (Lys(iPr)) is preferably protected by a PG selected from the group consisting of tert-butyloxycarbonyl (Boc), formyl (For), allyloxycarbonyl (Alloc) and benzyloxycarbonyl (Cbz); more preferably, the tert-butyloxycarbonyl (Boc) group is used;

the carbamoyl group (Cbm) of D-Aph(Cbm) is free or optionally protected with a PG, for instance with tert-butyl (tBu);

the p-amino group of p-amino-phenylalanine (Aph) is preferably protected by a PG selected from the group consisting of tert-butyloxycarbonyl (Boc), formyl (For), allyloxycarbonyl (Alloc) and benzyloxycarbonyl (Cbz); more preferably, the tert-butyloxycarbonyl (Boc) group is used;

the p-amino group of Aph, or of D-Aph, can be also masked as nitro group, thus needing reduction of nitro group of Phe(NO₂) or of D-Phe(NO₂) to amine at some stage in the preparation of degarelix of the present invention, and subsequent modification to introduce the dihydroorotyl moiety, to obtain Aph(Hor), or the carbamoyl moiety, to obtain D-Aph(Cbm).

Commercially available protected L- or D-amino acids are generally used. When not specified, the intended configuration at alpha-carbon is the L-configuration.

The Fmoc protected amino acids used as building blocks in the process of the present invention comprise Fmoc-D-Ala-OH, Fmoc-Pro-OH, Fmoc-Lys(iPr, PG)-OH, Fmoc-Leu-OH, Fmoc-D-Aph(Cbm)-OH, Fmoc-D-Aph(Cbm,PG)-OH, Fmoc-Aph(Hor)-OH, Fmoc-Ser(PG)-OH, Fmoc-D-Pal-OH, Fmoc-D-Cpa-OH, Fmoc-D-Nal-OH, Fmoc-Aph(PG)-OH, Fmoc-D-Aph(PG)-OH, Fmoc-Phe(NO₂)—OH and Fmoc-D-Phe(NO₂)—OH, wherein PG is a protective group as defined above.

In a preferred aspect of present invention, the coupling reactions in the preparation of degarelix of the present invention are performed in the presence of a coupling reagent.

Preferably, the coupling reagent is selected from the group consisting of N-hydroxysuccinimide (NHS), N,N′-diisopropylcarbodiimide (DIC), N,N′-dicyclohexylcarbodiimide (DCC), (Benzotriazol-1-yloxy)tripyrrolidinophosphonium hexafluorophosphate (PyBOP), 2-(7-Aza-1H-benzotriazole-1-yl)-1,1,3,3-tetramethyluronium hexafluorophosphate (HATU), 2-(1H-benzotriazole-1-yl)-1,1,3,3-tetramethyluronium hexafluorophosphate (HBTU), 2-(1H-Benzotriazole-1-yl)-1,1,3,3-tetramethylaminium tetrafluoroborate (TBTU) and ethyl-dimethylaminopropyl carbodiimide (EDC).

In a more preferred aspect of the present invention, the coupling reactions in the preparation of degarelix are performed in the presence of DIC.

More preferably, the reaction is carried out in the presence of N,N′-diisopropylcarbodiimide (DIC).

In a preferred aspect of present invention, the coupling reactions are performed also in the presence of an additive. The presence of an additive, when used in the coupling reaction, reduces loss of configuration at the carboxylic acid residue, increases coupling rates and reduces the risk of racemization.

Preferably, the additive is selected from the group consisting of 1-hydroxybenzotriazole (HOBt), 2-hydroxypyridine N-oxide, N-hydroxysuccinimide (NHS), 1-hydroxy-7-azabenzotriazole (HOAt), endo-N-hydroxy-5-norbornene-2,3-dicarboxamide and ethyl 2-cyano-2-hydroxyimino-acetate (OxymaPure). More preferably, the reaction is carried out in the presence of 2-cyano-2-hydroxyimino-acetate.

The coupling reactions in the preparation of degarelix of the present invention may optionally be performed in the presence of a detergent. The preferred detergents for the coupling via stepwise SPPS or via a convergent approach are non-ionic detergents, for instance Triton X-100 (also referred to as TX-100 or as polyethylene glycol tert-octylphenyl ether) or Tween 20, and most preferably Triton X-100. For instance, TX-100 may be used as 1% solution in DMF:DCM 50:50 v/v.

In a preferred aspect of present invention, the coupling reactions are performed in a solvent selected from the group consisting of DMF, DCM, THF, NMP, DMA or mixtures thereof. More preferably, the coupling is carried out in DMF.

In a preferred aspect of present invention, the coupling reactions are carried out at a temperature which can vary in the range 5-70° C., for instance in the range 5-40° C. Preferably, the temperature may vary in the range from room temperature (i.e. 15-20° C.) to 40° C., more preferably the temperature varies in the range 15-35° C., or even more preferably in the range of 15-25° C.

In the process of the present invention, the alpha-amino protecting groups, i.e. the Fmoc groups are cleaved by treatment with tert-butylamine (TBA). Tert-butylamine may be mixed with a suitable solvent, such as for instance DMF or DCM, or mixtures thereof; preferably DMF is used as a solvent. Also preferably, the concentration of tert-butylamine in the solvent varies in the range 5-50%, more preferably in the range 20-40%. Most preferred, Fmoc deprotection is carried out by using a 30% solution of tert-butylamine in DMF.

During Fmoc deprotection, a dibenzofulvene (DBF) byproduct forms during reaction. Fmoc cleavage in the process of the present invention may therefore be carried out in the optional presence of a DBF scavenger, such as DTT (dithiothreitol) or 1-octadecanethiol (C18SH).

Once the desired degarelix peptide has been obtained according to SPPS or CSPPS as described above and the Fmoc group has been cleaved from D-Nal, such N-terminal amino acid is acetylated at alpha-amino group by an acetylating agent, such as acetic acid, acetyl imidazole and acetic anhydride. Preferably, the reaction is carried out with acetic acid, in the presence of a coupling reagent, optionally with an additive, as defined above. More preferably, the acetylation is carried out with acetic acid, DIC and OxymaPure.

Finally, after the acetylated peptide sequence of degarelix is complete, the final deprotection and/or cleavage from the solid support is performed.

Preferably, such step is performed by using a specific mixture individualised for the resin used, in acidic or slightly acidic conditions, optionally in the presence of any scavenger.

Scavengers are substances, like, for instance, anisole, thioanisole, triisopropylsilane (TIS), 1,2-ethanedithiol and phenol, capable of minimize modification or destruction of the sensitive deprotected side chains, in the cleavage environment.

When a Wang resin is used, the treatment with a cleavage mixture, comprising TFA and any scavenger, provides both side-chains deprotection and cleavage from the resin. Such cleavage/deprotection step can be performed by using a mixture of TFA/thioanisole/anisole/dodecanethiol, for instance with a 90/5/2/3 (by volume) composition, or a mixture of TFA/water/phenol/TIS, for instance with 88/5/5/2 (by volume) composition, or any suitable mixture.

When a CTC resin is used, preferably in the preparation of a peptide fragment, the cleavage step can be performed by treatment with a mixture of HFIP:DCM (30:70 by volume) or 1-2 v/v % TFA solution in DCM. In particular, when the prepared peptide fragment is further subjected to a coupling, such cleavage does not remove the alpha-amino protecting group nor the side-chain protecting groups, thus yielding a full protected fragment, ready to react at its free C-terminal carboxylic acid.

When the process of the present invention is carried out by using a Rink Amide resin, a mixture of TFA and TIS may be used, for instance a mixture TFA/TIS/water (95/2.5/2.5 by volume). This treatment both removes any side-chain protection and cleaves the peptide from the resin.

When the process of the present invention involves a peptide synthesis in liquid phase, or a mixed liquid and solid phase preparation, all the features as above described apply mutatis mutandis. In particular, it is made reference to the coupling reactions conditions, comprising coupling reagents, additives, solvents, protective groups, Fmoc cleavage conditions, acetylations, which are easily adaptable in a clear manner by the person skilled in the art.

The crude final peptide obtained by cleavage from the resin or by last reaction in solution phase, i.e. crude degarelix, may then be optionally purified to increase its purity, for instance by preparative HPLC.

To this aim, a solution of the crude peptide is loaded onto an HPLC column with a suitable stationary phase, preferably C18 or C8 modified silica, and an aqueous mobile phase comprising an organic solvent, preferably acetonitrile or methanol, is passed through the column. A gradient of the mobile phase is applied, if necessary. The peptide with desired purity is collected and optionally lyophilized.

The present invention therefore provides a process for the preparation of degarelix (I), or a pharmaceutically acceptable salt thereof, by using Fmoc protected amino acids as building blocks, wherein the Fmoc group is cleaved by treatment with tert-butylamine.

In particular, the present invention provides a process for the preparation of degarelix (I), or a pharmaceutically acceptable salt thereof, by using Fmoc protected amino acids as building blocks, wherein at least after incorporation or formation of the orotyl residue of the peptide sequence, the Fmoc group is cleaved by treatment with tert-butylamine.

It is preferred that such process comprises stepwise synthesis on a solid support, which comprises an amino group linked to such support, wherein the steps comprise a) providing a solution of an amino acid or peptide whose alpha-amino group is protected by Fmoc; b) treating the solid support with such solution in the presence of at least a reagent for forming an amide bond between a carboxylic group of the dissolved amino acid or peptide and the alpha-amino group linked to the support for a time sufficient to form said amide bond, and c) cleaving Fmoc by treating the solid support with a base in an organic solvent, wherein the base is tert-Butylamine for at least those cleaving steps which follow the addition of an orotyl residue to the peptide, be it by incorporation of an Aph(Hor) into the peptide sequence, or by coupling of an orotyl residue on a Aph in position 5 of the peptide sequence.

In a preferred embodiment, the present invention provides a process for the preparation of degarelix which further comprises the use of one or more of the compounds selected from the group consisting of Fmoc-Aph(Hor)-OH, Fmoc-Phe(NO₂)—OH, Fmoc-D-Phe(NO₂)—OH and a peptide comprising one or more of Aph(Hor), Phe(NO₂) or D-Phe(NO₂).

In another preferred embodiment, the present invention provides a process for the preparation of degarelix which is performed by SPPS, which process comprises stepwise synthesis on a solid support, which comprises an amino group linked to such support, wherein at least the steps after incorporation or formation of the orotyl residue of the peptide sequence comprise:
a) providing a solution of an amino acid or peptide whose alpha-amino group is protected by Fmoc;
b) treating the solid support with such solution in the presence of at least a reagent for forming an amide bond between a carboxylic group of the dissolved amino acid or peptide and the alpha-amino group linked to the support for a time sufficient to form said amide bond, and
c) cleaving Fmoc by treating the solid support with tert-butylamine in an organic solvent.

In a preferred embodiment, the invention provides a process for the preparation of degarelix performed by SPPS as described above, wherein the orotyl residue has been incorporated by providing a solution of Fmoc-Aph(Hor)-OH; treating the solid support, which comprises an amino group linked to such support, with such solution in the presence of at least a reagent for forming an amide bond between a carboxylic group of the dissolved amino acid and the alpha-amino group linked to the support for a time sufficient to form said amide bond, and cleaving Fmoc by treating the solid support with tert-butylamine in an organic solvent.

The present invention further provides a process for the preparation of degarelix, as above defined, wherein in at least one step of the stepwise synthesis the solution treating the solid support comprises a reagent selected from the group consisting of Fmoc-Aph(Hor)-OH, Fmoc-Phe(NO₂)—OH, Fmoc-D-Phe(NO₂)—OH and a peptide comprising one or more of Aph(Hor), Phe(NO₂) or D-Phe(NO₂).

The incorporation of Fmoc-Phe(NO₂)—OH into the degarelix growing sequence in position 5 can be followed by reduction of the nitro group to amine and coupling with Hor to obtain Aph(Hor) before the addition of the subsequent amino acid in the sequence (i.e. Ser) or, more conveniently, such chemical transformation can be performed later on or at the end of the peptide elongation.

While the use of Fmoc-Phe(NO₂)—OH is followed by chemical transformation of the side-chain at some stage of the synthesis, the incorporation of Fmoc-Aph(Hor)-OH into the degarelix growing sequence in position 5 allows a straightforward synthesis of degarelix.

Analogously, the incorporation of Fmoc-D-Phe(NO₂)—OH into the degarelix growing sequence in position 6 can be followed by reduction of the nitro group to amine and then coupling with tert-butylisocyanate to obtain D-Aph(Cbm,tBu) before the addition of the subsequent amino acid in the sequence (e.g. Aph(Hor) or Phe(NO₂)); or, such chemical transformation of the side-chain can be performed later on or at the end of the peptide elongation.

Therefore, one embodiment of the present invention provides a process for the preparation of degarelix, or a pharmaceutically acceptable salt thereof, wherein such process comprises the steps of:

i) treating Fmoc-Phe(NO₂)-D-Aph(Cbm,PG)-Leu-Lys(iPr,PG)-Pro-D-Ala-X
with a reducing agent;
ii) reacting the resulting compound

### Fmoc-Aph-D-Aph(Cbm,PG)-Leu-Lys(iPr,PG)-Pro-D-Ala-X

with dihydroorotic acid, optionally in the presence of a coupling reagent,
and completing the preparation of degarelix on the obtained compound

### Fmoc-Aph(Hor)-D-Aph(Cbm,PG)-Leu-Lys(iPr,PG)-Pro-D-Ala-X

according to SPPS as described above,
wherein X is a solid support, preferably a Rink amide resin; and
PG is hydrogen (meaning that there is no protective group) or a protective group.

The reducing agent used to convert the nitro group into amine, e.g as in step i) above, can be for instance sodium dithionite, tin (II) chloride or iron powder. Preferably, the reduction is carried out in the presence of tin (II) chloride in a suitable solvent, for instance DMF, and in the presence of a base, like for instance DIPEA and DBU, preferably with DIPEA. Optionally the reduction reaction is performed in the presence of a nitrogen atmosphere.

The coupling reaction of dihydroorotic acid with a Fmoc protected peptide comprising Aph, e.g as in step b) above, is performed in the presence of a coupling reagent. Suitable coupling reagents are DCC, EDC and DIC. Preferably, the reaction is carried out in the presence of DIC. The reaction may also be carried out in the presence of a coupling reagent and an additive, which can be selected from the groups defined above. Preferably, the coupling with dihydroorotic acid is carried out in the presence of DIC and HOBt.

A further embodiment of the present invention provides a process for the preparation of degarelix, or a pharmaceutically acceptable salt thereof, wherein the chemical transformation of the nitro group is performed at the end of the peptide elongation.

Therefore, the present invention also provides a process for the preparation of degarelix, or a pharmaceutically acceptable salt thereof, wherein such process comprises the steps of treating the compound Fmoc-Phe(NO₂)-D-Aph(Cbm,PG)-Leu-Lys(iPr,PG)-Pro-D-Ala-X with tert-butylamine;

d) completing the preparation of degarelix on the obtained compound

### Phe(NO₂)-D-Aph(Cbm,PG)-Leu-Lys(iPr,PG)-Pro-D-Ala-X

according to SPPS as described above;
e) acetylating the obtained decapeptide

### D-Nal-D-Cpa-D-Pal-Ser(PG)-Phe(NO₂)-D-Aph(Cbm,PG)-Leu-Lys(iPr,PG)-Pro-D-Ala-X

in the presence of an acetylating agent;
f) treating the resulting compound

### Ac-D-Nal-D-Cpa-D-Pal-Ser(PG)-Phe(NO₂)-D-Aph(Cbm,PG)-Leu-Lys(iPr,PG)-Pro-D-Ala-X

with a reducing agent;
g) reacting the resulting compound

### Ac-D-Nal-D-Cpa-D-Pal-Ser(PG)-Aph-D-Aph(Cbm,PG)-Leu-Lys(iPr,PG)-Pro-D-Ala-X

with dihydroorotic acid, optionally in the presence of a coupling reagent, to obtain

### Ac-D-Nal-D-Cpa-D-Pal-Ser(PG)-Aph(Hor)-D-Aph(Cbm,PG)-Leu-Lys(iPr,PG)-Pro-D-Ala-X

i.e. protected degarelix attached to a solid support, which is then treated as described above to finally obtain crude degarelix,
and wherein X and PG are as defined above.

The reducing agent used to convert the nitro group into amine, e.g as in step f) above, can be for instance sodium dithionite, tin (II) chloride or iron powder. Preferably, the reduction is carried out in the presence of tin (II) chloride in a suitable solvent, for instance DMF, and in the presence of a base, like for instance DIPEA and DBU, preferably with DIPEA. Optionally the reduction reaction is performed in the presence of a nitrogen atmosphere.

The above described process is exemplified in Example 4 of present disclosure.

Therefore, the present invention provides a process for the preparation of degarelix as defined above, wherein the solid support before treatment with tert-butylamine for Fmoc group cleavage (or obtained in step b) comprises:

### Fmoc-D-Ala-X,

### Fmoc-Pro-D-Ala-X,

### Fmoc-Lys(iPr,PG)-Pro-D-Ala-X,

### Fmoc-Leu-Lys(iPr,PG)-Pro-D-Ala-X,

### Fmoc-W-Leu-Lys(iPr,PG)-Pro-D-Ala-X,

### Fmoc-Z-W-Leu-Lys(iPr,PG)-Pro-D-Ala-X,

### Fmoc-Ser(PG)-Z-W-Leu-Lys(iPr,PG)-Pro-D-Ala-X,

### Fmoc-D-Pal-Ser(PG)-Z-W-Leu-Lys(iPr,PG)-Pro-D-Ala-X,

### Fmoc-D-Cpa-D-Pal-Ser(PG)-Z-W-Leu-Lys(iPr,PG)-Pro-D-Ala-X, or

### Fmoc-D-Nal-D-Cpa-D-Pal-Ser(PG)-Z-W-Leu-Lys(iPr,PG)-Pro-D-Ala-X,

wherein
X is a solid support, preferably a Rink amide resin;

### Z is Aph(Hor), Aph(PG), or Phe(NO₂);

### W is D-Aph(Cbm,PG), D-Aph(PG), or D-Phe(NO₂); and

PG is hydrogen (meaning that there is no protective group) or a protective group and which results in degarelix.

Degarelix prepared by the process(es) of the present invention is characterized by an impurity profile which allows for a more effective purification via the standard purification method, HPLC purification.

The present invention provides a process for the preparation of degarelix, wherein degarelix comprises 0.5% by weight or less, e.g., 0.3% by weight or less, 0.15% by weight or less, 0.1% by weight or less, or 0.05% by weight or less, of hydantoin-degarelix impurity (II). In other embodiments, the present invention provides a process for the preparation of degarelix, wherein degarelix comprises 0.05%-0.5% by weight of hydantoin-degarelix impurity (II), e.g., 0.05%-0.4%, 0.05%-0.3%, 0.05%-0.15%, 0.1%-0.5%, or 0.1%-0.3%, by weight of hydantoin-degarelix impurity (II).

The preferred embodiments of the invention provide a process for the preparation of degarelix, wherein degarelix comprises 0.15% by weight or less, 0.1% by weight or less, or 0.05% by weight or less, of hydantoin-degarelix impurity (II). Even more preferred is a process for the preparation of degarelix, wherein degarelix comprises 0.05%-0.15% by weight of hydantoin-degarelix impurity (II).

### Abbreviations

Aph p-amino-phenylalanine
Amf p-aminomethyl-phenylalanine
Atz 3′-amino-1H-1′,2′,4′-triazol-5′-yl, 5
Cbm Carbamoyl
For Formyl
Imz 2-imidazolidone-4-carbonyl
h hour
min minutes
GnRH Gonadotropin releasing hormone
SPPS Solid phase peptide synthesis
LPPS Liquid phase peptide synthesis
MBHA resin Methyl benzhydryl amide resin
Fmoc Rink amide resin 4-(2′,4′-Dimethoxyphenyl-Fmoc-aminomethyl)-phenoxyacetamidomethyl polystyrene resin
Fmoc Rink amide MBHA resin 4-(2′,4′-Dimethoxyphenyl-Fmoc-aminomethyl)-phenoxyacetamido-4-methylbenzhydrylamine polystyrene resin
Fmoc Rink amide AM resin 4-(2′,4′-Dimethoxyphenyl-Fmoc-aminomethyl)-phenoxyacetamido-aminomethyl resin
Fmoc-D-Ala-Rink resin 9-Fluorenylmethyloxycarbonyl-D-alanine-Rink resin
Fmoc-D-Ala-OH 9-Fluorenylmethyloxycarbonyl-D-alanine
Fmoc-Pro-OH 9-Fluorenylmethyloxycarbonyl-L-proline
Fmoc-Lys(iPr, Boc)-OH 9-Fluorenylmethyloxycarbonyl-N(ε)-isopropyl-N(ε)-Boc-lysine
Fmoc-Leu-OH 9-Fluorenylmethyloxycarbonyl-leucine-OH
Phe(NO₂) L-4-nitrophenylalanine
D-Phe(NO₂) D-4-nitrophenylalanine
Fmoc-D-Phe(NO₂)—OH Fluorenylmethoxycarbonyl-D-4-nitrophenylalanine
Fmoc-Phe(NO₂)—OH Fluorenylmethoxycarbonyl-4-L-nitrophenylalanine
Fmoc-D-Aph(Cbm)-OH 9-Fluorenylmethyloxycarbonyl-N(4)-carbamoyl-D-4-aminophenylalanine
Fmoc-Ser(tBu)-OH 9-Fluorenylmethyloxycarbonyl-O-t-butyl-serine
Fmoc-D-Pal-OH 9-Fluorenylmethyloxycarbonyl-D-3-pyridylalanine
Fmoc-D-Cpa-OH/Fmoc-D-Phe(4-Cl)—OH 9-Fluorenylmethyloxycarbonyl-D-4-chlorophenylalanine
Fmoc-D-Nal-OH 9-Fluorenylmethyloxycarbonyl-D-2-naphtylalanine
Fmoc-Aph(Hor)-OH 9-Fluorenylmethyloxycarbonyl-N(4)-(L-hydroorotyl)-4-aminophenylalanine
Aph(Hor) N(4)-(L-hydroorotyl)-4-aminophenylalanine
D-Aph(Cbm) 4-(Aminocarbonyl)amino-D-Phenylalanine
Aph(Trt) 4-(trityl)amino-D-Phenylalanine
Hor Dihydroorotyl moiety
Hor-OH (L)dihydroorotic acid
Fmoc 9-Fluorenylmethyloxycarbonyl
Boc t-Butyloxycarbonyl
Dde 1,1-Dichloro-2,2-bis(p-chlorophenyl)ethylene
HPLC High performance liquid chromatography
DIPEA Diisopropylethylamine
tBu-NCO tert-butyl isocyanate
Ac₂O Acetic anhydride
SnCl₂ Tin (II) chloride
Hor-OH (L)Dihydroorotic acid
HOBt 1-Hydroxybenzotriazole
TFA Trifluoroacetic acid
DMF N,N-dimethylformamide
DMA N,N-dimethylacetamide
NMP N-methylpyrrolidone
THF Tetrahydrofuran
DCM Dichloromethane
DCC N,N′-dicyclohexylcarbodiimide
EDC 1-Ethyl-3-(3-dimethylaminopropyl) carbodiimide
DIC Diisopropylcarbodiimide
HBTU 2-(1H-benzotriazol-1-yl)-1,1,3,3-tetramethyluronium hexafluorophosphate
HATU 2-(7-Aza-1H-benzotriazole-1-yl)-1,1,3,3-tetramethyluronium hexafluorophosphate
TBTU 2-(1H-Benzotriazole-1-yl)-1,1,3,3-tetramethylaminium tetrafluoroborate
TIS tri-isopropylsilane
HFIP Hexafluoro-2-propanol
OxymaPure Ethyl 2-cyano-2-hydroxyimino-acetate

### EXAMPLES

Detailed experimental parameters suitable for the preparation of degarelix according to the present invention are provided by the following examples, which are intended to be illustrative and not limiting of all possible embodiments of the invention.

Unless otherwise noted, all materials, solvents and reagents were obtained from commercial suppliers, of the best grade, and used without further purification.

Solid-phase synthesis of the peptides was carried out using common peptide synthesizers, such as Biotage Syrowave instrument (automated syntheses) and Biotage MultiSynTech (semi automated syntheses).

HPLC analyses were performed on Agilent Technologies 1200 or 1290 Infinity II instruments, using columns C8 Zorbax Eclipse Plus (4.6×50 mm, 1.8 μm) or Waters Aquity UPLC BEH C18 (150 mm×3 mm; 1.7 μm), respectively. The molar yields (%) are calculated considering the final moles obtained (based on Assay) divided by the initial moles. Assays (%) are calculated by HPLC, comparing the peak area of the sample with the peak area of the standard.

### Example 1: General Procedure for Stability Experiments of Degarelix in the Presence of Organic Bases: Screening of DBU, Pyrrolidine, Piperidine, TBA, N-Methyl-Piperazine and Morpholine

Purified degarelix with hydantoin-degarelix impurity (II) content <0.15% was dissolved in a mixture of DMF at room temperature and the selected amine, in order to obtain 130 mg/ml peptide concentration. Aliquots of the solution were analyzed by HPLC after 20 min, 1 h 40 min, and 20 h.

In parallel, stability of degarelix was tested after addition of 5% water to each sample.

Results are reported in Table 1 of the description as HPLC peak area % of the hydantoin-degarelix impurity (II).

### Example 2: General Procedure for the Fmoc Deprotection Kinetics Study: Screening of Piperidine, TBA, N-Methylpiperazine and Morpholine

10 mg of Fmoc protected Rink amide resin (Fmoc-Phe(p-NO₂)-Rink Amide Resin, Fmoc-Rink Amide Resin or Fmoc-Ser(tBu)-Rink Amide Resin) were swollen in DMF for 15 min and the selected amine was added to the suspension in order to achieve the desired concentration (20% piperidine, 30% TBA, 5% N-methylpiperazine or 50% morpholine) in the final 1 ml deprotection mixture total volume. The reaction mixture was stirred at room temperature and samples of the solution (10 μL) were taken after 20 min, 1 h 40 min and 20 h. The samples were diluted with 990 μL of DMF in 1 cm quartz cuvette. The absorbance was measured at 301 nm and the loading was calculated by formula

L=(A₃₀₁×V×d)/(K×w×M)

where L is the resin loading, A₃₀₁ is absorbance at 301 nm, V is volume of the cleavage solution, K is the extinction coefficient (7800 mL/(mmol×cm)), w is the optical path length, M is the exact weight of the resin sample (in grams), d is the dilution factor (100 for each experiment).

The % Fmoc removal values (i.e. normalized absorbance measurements) are reported in FIG. 1 vs. time(min), for Fmoc-Phe(p-NO₂)-Rink Amide Resin.

### Example 3: Stepwise SPPS of Degarelix

The synthesis was carried out by using Fmoc Rink amide resin (250 mg, loading 0.65 mmol/g). After swelling of the resin in 2 ml of DMF, Fmoc protective group was removed by 30% solution of tert-butylamine in DMF (2×2 ml, 5 min and 20 min) and the resin was washed with DMF (4×2 ml). Fmoc-D-Ala-OH, Fmoc-Pro-OH, Fmoc-Lys(iPr,Boc)-OH, Fmoc-Leu-OH, Fmoc-D-Aph(Cbm)-OH, Fmoc-Aph(Hor)-OH, Fmoc-Ser(tBu)-OH, Fmoc-D-Pal-OH, Fmoc-D-Cpa-OH, Fmoc-D-Nal-OH (three-fold excess with respect to the loading of the resin) were pre-activated by DIC and OxymaPure (three-fold excess of the reagents with respect to the loading of the resin) for 3 min and coupled to the resin in 60 min. In case of Fmoc-Aph(Hor)-OH the coupling time was increased to 3 h. After each coupling step the Fmoc protective group was removed by treating the peptide resin with a 30% solution of tert-butylamine in DMF (2×2 ml, 5 min and 20 min) and the resin was washed with DMF (4×2 ml). The N-terminal amino group was acetylated with acetic acid pre-activated with the mixture of DIC and OxymaPure (three-fold excess of the reagents with respect to the loading of the resin). Then the peptide resin was washed with DMF (3×2 ml) and DCM (3×2 ml). Dry peptide resin was suspended in 3 ml of the mixture TFA/TIS/water (95/2.5/2.5 v/v/v) and stirred for 4 h. The resin was filtered off and methyl tert-butyl ether (10 ml) cooled to 4° C. was added to the solution. The peptide was filtered and dried in vacuo to obtain 265 mg (assay 50%) crude degarelix with an HPLC purity of 87.5% and hydantoin-degarelix impurity (II)<0.15%. Molar yield 50%.

### Example 4: SPPS of Degarelix Via Phe(NO₂) Reduction

The synthesis was carried out by using Fmoc Rink amide resin (250 mg, loading 0.65 mmol/g). After swelling of the resin in 2 ml of DMF, Fmoc protective group was removed by 30% solution of TBA in DMF (2×2 ml, 5 min and 20 min) and the resin was washed with DMF (4×2 ml). Fmoc-D-Ala-OH, Fmoc-Pro-OH, Fmoc-Lys(iPr, Boc)-OH, Fmoc-Leu-OH, Fmoc-D-Aph(Cbm)-OH, Fmoc-Phe(NO₂)—OH, Fmoc-Ser(tBu)-OH, Fmoc-D-Pal-OH, Fmoc-D-Cpa-OH, Fmoc-D-Nal-OH (three-fold excess with respect to the loading of the resin and two-fold excess in case of Fmoc-Lys(iPr, Boc)-OH) were pre-activated by DIC and OxymaPure (three-fold excess of the reagents with respect to the loading of the resin) for 3 min and coupled to the resin for 90 min. After each coupling the unreacted amino groups, as well as the N-terminal amino group of D-Nal, were capped using 2 ml of the solution of acetic anhydride (1 ml) and DIPEA (2 ml) in 7 ml of DMF. After each capping step the Fmoc protective group was removed by treating the peptide resin with a 30% solution of tert-butylamine in DMF (2×2 ml, 5 min and 20 min) and the resin was washed with DMF (4×2 ml). The obtained peptide resin was treated with a solution of SnCl₂ (10 eq) and DIPEA (1.2 eq) in 2.5 ml of DMF for 15 h under nitrogen. At the end of the reaction, the solvent was filtered off and the resin was washed with DMF (5×2 ml). A solution of Hor-OH (1.5 eq), DIC (1.5 eq) and HOBt (1.5 eq) in 2.5 ml of DMF was added to the resin. After 1.5 h the solvent was filtered off and freshly prepared mixture of Hor-OH, DIC, HOBt was added. The reaction continued for further 1.5 h. Then the peptide resin was washed with DMF (3×2 ml) and DCM (3×2 ml). Dry peptide resin was suspended in 3 ml of the mixture TFA/TIS/water (95/2.5/2.5 v/v/v) and stirred for 4 h. The resin was filtered off and methyl tert-butyl ether (10 ml) cooled to 4° C. was added to the solution. The peptide was filtered and dried in vacuo to obtain 303 mg (assay 52%) crude degarelix with an HPLC purity of 88.6% and hydantoin-degarelix impurity (II)<0.15%. Molar yield 55%.

### Example 5: Fmoc Deprotection Kinetics Study with TBA at Different Concentrations on Two Substrates: Fmoc-Rink Amide Resin and Fmoc-Ser(tBu)-Rink Amide Resin

10 mg of Fmoc protected Rink amide resin or Fmoc-Ser(tBu)-Rink Amide Resin were swollen in DMF for 15 min and TBA was added to the suspension in order to achieve the desired concentration (5%, 10%, 15%, 20%, 30%, 40%, and 50%) in the final 1 ml deprotection mixture total volume (seven samples for each substrate). The reaction mixture was stirred at room temperature and samples of the solution (10 μL) were taken after 0.5, 1, 5, 10, 15, 20, and 30 min. The samples were diluted with 990 μL of DMF in 1 cm quartz cuvette. The absorbance was measured at 301 nm and the loading was calculated as described in Example 2.

The % Fmoc removal values are reported in FIGS. 2 and 3 vs. time(min), for Fmoc-Rink amide resin and Fmoc-Ser(tBu)-Rink amide resin, respectively.

1. A process for the preparation of the peptide degarelix (I), or a pharmaceutically acceptable salt thereof, said method comprising 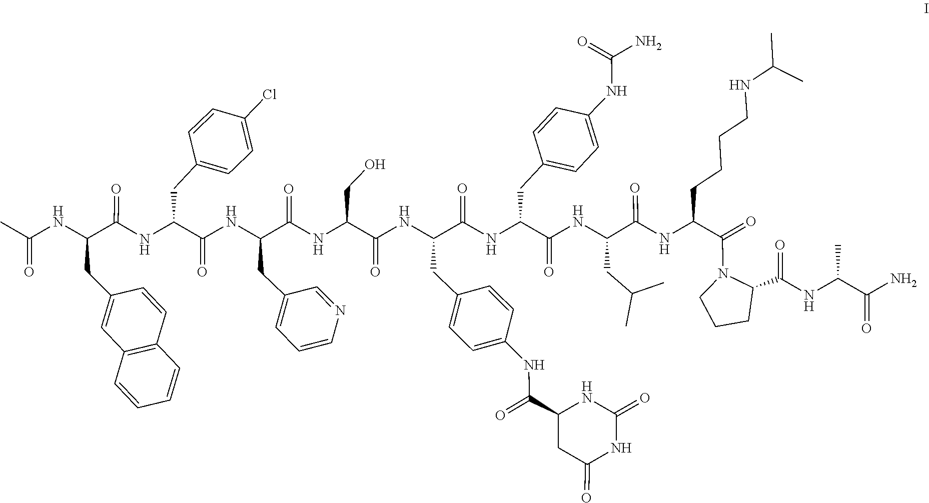 [SMILES: CC(=O)N[C@H](Cc1ccc2ccccc2c1)C(=O)N[C@H](Cc1ccc(Cl)cc1)C(=O)N[C@H](Cc1cccnc1)C(=O)N[C@@H](CO)C(=O)N[C@@H](Cc1ccc(NC(=O)[C@@H]2CC(=O)NC(=O)N2)cc1)C(=O)N[C@H](Cc1ccc(NC(N)=O)cc1)C(=O)N[C@@H](CC(C)C)C(=O)N[C@@H](CCCCNC(C)C)C(=O)N1CCC[C@H]1C(=O)N[C@H](C)C(N)=O]
using Fmoc protected amino acids as building blocks, wherein the Fmoc group is cleaved by treatment with tert-butylamine.

2. The process according to claim 1, wherein at least after incorporation or formation of an orotyl residue into the peptide sequence the Fmoc group is cleaved by treatment with tert-butylamine.

3. The process according to claim 1, wherein such process is performed by solid phase peptide synthesis.

4. The process according to claim 1, wherein such process comprises stepwise synthesis on a solid support, which comprises an amino group linked to such support, wherein the steps comprise:
a) providing a solution of an amino acid or peptide whose alpha-amino group is protected by a Fmoc group;
b) treating the solid support with such solution in the presence of at least a reagent for forming an amide bond between a carboxylic group of the dissolved amino acid or peptide and the alpha-amino group linked to the support for a time sufficient to form said amide bond, and
c) cleaving the Fmoc group by treating the solid support with a base in an organic solvent, wherein the base is tert-butylamine.

5. The process according to claim 4, wherein the base is tert-butylamine in those steps following incorporation of an orotyl residue into the peptide, or formation of such an orotyl residue on the peptide, linked to the solid support.

6. The process according to claim 4, wherein an orotyl residue has been incorporated by providing a solution of Fmoc-Aph(Hor)-OH or of a peptide comprising Aph(Hor), treating the solid support with such solution in the presence of at least a reagent for forming an amide bond between a carboxylic group of the dissolved amino acid or peptide and the alpha-amino group linked to the solid support for a time sufficient to form said amide bond, and cleaving the Fmoc group by treating the solid support with tert-butylamine.

7. The process according to claim 1, wherein such process comprises the use of one or more compounds selected from the group consisting of Fmoc-Phe(NO₂)—OH, Fmoc-D-Phe(NO₂)—OH and a peptide comprising Phe(NO₂) or D-Phe(NO₂).

8. The process according to claim 3, wherein the solid support before treatment with tert-butylamine for Fmoc group cleavage—or obtained in step b)—comprises:
Fmoc-D-Ala-X,
Fmoc-Pro-D-Ala-X,
Fmoc-Lys(iPr,PG)-Pro-D-Ala-X,
Fmoc-Leu-Lys(iPr,PG)-Pro-D-Ala-X,
Fmoc-W-Leu-Lys(iPr,PG)-Pro-D-Ala-X,
Fmoc-Z-W-Leu-Lys(iPr,PG)-Pro-D-Ala-X,
Fmoc-Ser(PG)-Z-W-Leu-Lys(iPr,PG)-Pro-D-Ala-X,
Fmoc-D-Pal-Ser(PG)-Z-W-Leu-Lys(iPr,PG)-Pro-D-Ala-X,
Fmoc-D-Cpa-D-Pal-Ser(PG)-Z-W-Leu-Lys(iPr,PG)-Pro-D-Ala-X, or
Fmoc-D-Nal-D-Cpa-D-Pal-Ser(PG)-Z-W-Leu-Lys(iPr,PG)-Pro-D-Ala-X,
wherein
X is a solid support;
Z is Aph(Hor), Aph(PG), or Phe(NO₂);
W is D-Aph(Cbm,PG), D-Aph(PG), or D-Phe(NO₂); and
PG is hydrogen or a protective group.

9. The process according to claim 4, wherein the solid support before treatment with tert-butylamine for Fmoc group cleavage—or obtained in step b)—comprises:
Fmoc-Phe(NO₂)-D-Aph(Cbm,PG)-Leu-Lys(iPr,PG)-Pro-D-Ala-X, or
Fmoc-Aph(Hor)-D-Aph(Cbm,PG)-Leu-Lys(iPr,PG)-Pro-D-Ala-X.

10. The process according to claim 7, further comprising the steps of:
i) treating Fmoc-Phe(NO₂)-D-Aph(Cbm,PG)-Leu-Lys(iPr,PG)-Pro-D-Ala-X
with a reducing agent;
ii) reacting the resulting compound
Fmoc-Aph-D-Aph(Cbm,PG)-Leu-Lys(iPr,PG)-Pro-D-Ala-X
with dihydroorotic acid, optionally in the presence of a coupling reagent,
and completing the preparation of degarelix on the obtained compound
Fmoc-Aph(Hor)-D-Aph(Cbm,PG)-Leu-Lys(iPr,PG)-Pro-D-Ala-X
according to SPPS;
wherein X is a solid support; and
PG is hydrogen or a protective group.

11. The process according to claim 7, wherein in step c) the compound
Fmoc-Phe(NO₂)-D-Aph(Cbm,PG)-Leu-Lys(iPr,PG)-Pro-D-Ala-X is treated with tert-butylamine, further comprising:
d) completing the preparation of degarelix on the obtained compound
Phe(NO₂)-D-Aph(Cbm,PG)-Leu-Lys(iPr,PG)-Pro-D-Ala-X
according to SPPS;
e) acetylating the obtained
D-Nal-D-Cpa-D-Pal-Ser(PG)-Phe(NO₂)-D-Aph(Cbm,PG)-Leu-Lys(iPr,PG)-Pro-D-Ala-X in the presence of an acetylating agent;
f) treating the resulting compound
Ac-D-Nal-D-Cpa-D-Pal-Ser(PG)-Phe(NO₂)-D-Aph(Cbm,PG)-Leu-Lys(iPr,PG)-Pro-D-Ala-X
with a reducing agent;
g) reacting the resulting compound
Ac-D-Nal-D-Cpa-D-Pal-Ser(PG)-Aph-D-Aph(Cbm,PG)-Leu-Lys(iPr,PG)-Pro-D-Ala-X
with dihydroorotic acid, optionally in the presence of a coupling reagent, to obtain
Ac-D-Nal-D-Cpa-D-Pal-Ser(PG)-Aph(Hor)-D-Aph(Cbm,PG)-Leu-Lys(iPr,PG)-Pro-D-Ala-X;
wherein X is a solid support; and
PG is hydrogen or a protective group.

12. The process according to claim 1, wherein degarelix or its pharmaceutically acceptable salt, comprises 0.15% by weight or less of hydantoin-degarelix impurity (II).

13. The process according to claim 1, wherein the concentration of tert-butylamine is in the range 5 to 50%, and wherein Fmoc group cleavage is performed in the organic solvent DMF.

14. The process according to claim 1, wherein the reagent for forming an amide bond comprises diisopropylcarbodiimide.

15. The process according to claim 1, wherein the temperature of Fmoc group cleavage is in the range 5 to 40° C.

16. The process according to claim 14, wherein the reagent for forming an amide bond further comprises an additive selected from the group consisting of 1-hydroxybenzotriazole, 2-hydroxypyridine N-oxide, N-hydroxysuccinimide, 1-hydroxy-7-azabenzotriazole, endo-N-hydroxy-5-norbornene-2,3-dicarboxamide and ethyl 2-cyano-2-hydroxyimino-acetate.

17. Degarelix prepared using Fmoc protected amino acids as building blocks, wherein the Fmoc group is cleaved by treatment with tert-butylamine.

18. The method according to claim 3, wherein said solid phase peptide synthesis is performed on a solid support chosen from Rink amide, Rink amide AM and Rink amide MBHA resin.

19. The method according to claim 8, wherein said X is a Rink amide resin.

20. The method according to claim 9, wherein said reducing agent is used with tin chloride (II), or in the presence of DIPEA in DMF.
